(19) United States
(12) Patent Application Publication
(10) Pub. No.: US 2013/0237933 A1
(43) Pub. Date: Sep. 12, 2013

(54) TAMPON STRUCTURE

(71) Applicant: FENG CHIA UNIVERSITY, Taichung City (TW)

(72) Inventor: TSE-HAO KO, Taichung City (TW)

(73) Assignee: FENG CHIA UNIVERSITY, Taichung City (TW)

(21) Appl. No.: 13/669,407

(22) Filed: Nov. 5, 2012

(30) Foreign Application Priority Data
Mar. 12, 2012 (TW) 101108266

(51) Int. Cl. A61F 13/84; A61F 13/20

(52) U.S. Cl. CPC A61F 13/8405; A61F 13/2051
USPC 604/286; 604/360; 604/367; 604/375; 604/377; 604/374

(57) ABSTRACT

The present invention relates to a tampon structure, the tampon structure comprise at least one absorbent carbonaceous base with at least two precious metals, an absorber, a surface layer, and a rope. The copper ions and silver ions can be released by the at least two precious metals in aqueous solution to reach the health effects of the female vagina.

### BACKGROUND OF THE INVENTION

1. Field of the Invention

The present invention relates in general to a tampon structure comprising at least one absorbent carbonaceous base and at least two precious metals and more particularly to the ability of inhibiting the growth of microbes/germs and mold and absorbing the vaginal discharge and unpleasant odor.

2. Description of Related Art

The female's pudendal infection and inflammation are generally called vaginitis.

The common symptom of vaginitis includes: itching, red, swell, pain, calor, and abnormally increasing vaginal discharge. The possible reasons are due to infection, allergy, stimulation, improper sanitation or personal hygiene, the variation of pH value or normal flora in vagina. There are three types of vaginitis including: bacterial vaginosis accounting for 40-50%, vulvovaginal candidiasis accounting for 20-25%, and trichomonas vaginitis accounting for 15-20%. Other reasons may be the multi-infection, infection of pinworm and non-infected reasons.

To avoid the vaginitis, there are several ways can be done; keeping regular lifestyle, balanced diet, good personal hygiene, wearing clothes with fabric that breathe well, avoiding improper vaginal irrigation. Because Taiwan settles in a subtropical area which has hot and wet climate and longer summer season, and female there is busy with work and study which causing the irregular lifestyle, it is easy to have vaginitis.

Furthermore, tampons and pads used in female's period cause the external pudendum with moisture and heat. The tampon with blood is a major reason for germs to grow which causing unpleasant odor and raising the possibility of vaginitis.

By and large, treatments for vaginitis include wet dressing, warm sitz bathing, giving anti-viral drug, laser treatment, frozen surgery, giving antibiotics like penicillin or tetracycline, suppository, and ointment. In the publication of Sexually Transmitted Diseases Treatment Guidelines, 2006, it indicated that the medication for bacterial vaginosis and vulvovaginal candidiasis is metronidazole, butoconazole, clotrimazole, miconazole or nystatin. However, there are drawbacks of described treatments such as the possibility of drug resistance because of taking antibiotics, difficulties with using suppository, and applying regional ointment by individual. In addition, it usually takes more than 10 days for taking antibiotics or other drugs in one treatment, or it usually takes more than one time for frozen surgery. Since infections of vagina are the most common reasons for women asking for treatments in gynecological clinic, aforementioned drawbacks of the treatments should be solved.

### SUMMARY OF THE INVENTION

The objective of the present invention is to provide a tampon structure comprising: at least one absorbent carbonaceous base carrying at least two precious metals, an absorber, a surface layer and a rope.

Another objective of the present invention is to provide the tampon structure which can treat bacterial vaginosis and fugal vaginitis. The tampon structure comprises at least one absorbent carbonaceous base which the at least two precious metals are carried by and/or inserted in. The at least one absorbent carbonaceous base is chosen from powder of active carbon, particle of active carbon, powder of bamboo charcoal, active carbon fiber, natural graphite, flexible graphite, expandable graphite, carbon nanotube and the combination thereof The at least two precious metals are chosen from copper oxide which can release monovalent copper ions or divalent copper ions in a solution, and silver particles which can release silver ion in a solution. Purposes of vaginal healthcare and increasing the efficiency of wound healing are achieved because of the release of the monovalent copper ions or the divalent copper ions from a moisture environment to inhibit the growth of fungus and the release of the silver ion in moisture environment to kill microbes/germs. For enhancing the anti-bacteria and anti-fungus effects, except for the copper oxide and silver particles on the at least one absorbent carbonaceous base, other metals can be gold, palladium, platinum, aluminum, nickel, cobalt, zinc, silicon, calcium, titanium, chromium, manganese, and the combination thereof The combination can comprise other therapeutically active components depending on needs.

Characteristics of the at least one absorbent carbonaceous based on the present invention are the following: the surface area analysis by using Brunauer-Emmett-Teller (BET) method, the BET value is over 400 m²/g, the real density is over 1.6 g/ml, the carbon content is over 75 wt %, the oxygen content is 0.1-0.25 wt %, and average pore diameter is 0.5-10 nm by using the BET method. The type of the at least one absorbent carbonaceous base can be cloth, nonwoven, paper, felt, and the combination thereof The average diameter of the particles of the at least two precious metals carried by and/or inserted in the at least one absorbent carbonaceous base is less than 2000 nm; the best is less than 500 nm. Content of the at least two precious metals accounts for less than 5 wt % of the at least one absorbent carbonaceous base; the best at least two precious metals' content accounts for less than 1 wt % of the at least one absorbent carbonaceous base.

For increasing the volume of absorption, it is allowed to add superabsorbent polymer or highly absorbent natural fiber, for example, cotton, linen, wool; synthetic fiber, wooden renewable cotton, and the combination thereof into the internal layer of the at least one absorbent carbonaceous base.

The application of the present invention is within the range of the feminine hygiene products. Moreover, it is for reducing the possibility of vaginal infection of during the females' period by inhibiting the growth of the bacteria and fungus and for absorbing and eliminating the possible odor. In addition, the function of inhibiting bacteria and fungus in the present invention can be achieved without any drug or antibiotics, the tampon structure can avoid the drug resistance to the antibacterial or antifungal drug. Moreover, it can solve the inconvenience and danger of those women having doubts and problems of choosing drug such as pregnant women or women who are allergic to some drugs. To further enhance the effect of antibacterial and antifungal treatment, some therapeutically active components can be added in depending on the needs. The therapeutically active components could comprise following components but not for the limitation such as metronidazole, clindamycin, butoconazole, clorimazole, miconazole, nystatin tioconazole, terconazole, econazole, erythromycin, tetrachcline, cephalosporin, acrisorcin, haloprogin, iodochlorhydroxyquin, tolnaftate, triacetin, centella asiatica, econazole nitrate, mafenide, mupirocin, povidone iodine, and the combination thereof.

The tampon structure in this present invention can also be used by postpartum women. Applying to the birth canal or adjacent tissue, the tampon structure of this invention can also increase the efficiency of wound healing.

The better choices for the at least one absorbent carbonaceous based on this present invention can be active carbon, active carbon fiber, porous graphite, flexible graphite, expandable graphite, and the combination thereof Because the active carbon possesses the characteristics of massive absorption power and kinds of forces like van der waals force, it can absorb microbes/germs on the at least one absorbent carbonaceous base effectively. Accordingly, the at least two precious metals carried by and/or inserted in the at least one absorbent carbonaceous base can display the effect on the antibacterial and antifungal function easily. The microbes/germs and fungus are reduced due to the antibacterial and antifungal function from the at least two precious metals. Consequently, the possibility of odor and infection are also reduced. The odor and the vaginal discharge are absorbed on the at least one absorbent carbonaceous base and the absorber making the odor be eliminated. Moreover, the originally existing microbes/germs, fungus, mold, vaginal discharge in the vagina can be absorbed on the aforementioned at least one absorbent carbonaceous base and the absorber. When the at least one absorbent carbonaceous base and the absorber are removed, the microbes/germs, fungus, mold and vaginal discharge are also removed with the at least one absorbent carbonaceous base and the absorber. Therefore, the possibility of infection is reduced, the symptom of infection is alleviated and the odor is avoided. Because of the described characteristics, the applying of the tampon structure of this invention on the birth canal or adjacent tissue of wound can make the wound difficult to be ulcerated and inflamed. Due to the antibacterial and antifungal ability, the possibility of infection is reduced. Consequently, it is faster for the wound to be healed well.

### BRIEF DESCRIPTION OF THE DRAWINGS

FIG. 1 illustrates one embodiment of the present invention.

FIG. 2 illustrates another embodiment of the present invention.

FIG. 3 illustrates the other embodiment of the present invention.

FIG. 4a and FIG. 4b illustrate one more embodiment of the present invention.

### DETAILED DESCRIPTION OF THE INVENTION

The present invention may best be understood by reference to the following description in conjunction with the accompanying drawings. Embodiments of the present invention are as shown in FIG. 1 to FIG. 4b. A tampon structure of this invention comprises an absorber 300, at least one absorbent carbonaceous base 200 covering on the absorber 300, a surface layer 100, and a rope 400. Moreover, the tampon structure can be column shape or strip shape. The better choices of the at least one absorbent carbonaceous base 200 are from the following materials but not for limitation such as active carbon, active carbon fiber, porous graphite, flexible graphite, expandable graphite, and the combination of thereof. The types of the at least one absorbent carbonaceous base 200 are the following types but not for limitation such as active carbon paper, paper made of bamboo charcoal, active carbon fiber cloth, active carbon fiber paper, active carbon fiber felt, and the combination of thereof For the reduction of the cost, the at least one absorbent carbonaceous base 200 can be mixed with natural fibers or synthetic fibers to make the type of non-woven, non-woven cloth or paper. The at least one absorbent carbonaceous base 200 which at least two precious metals 201 are carried by and/or inserted in. The at least two precious metals 201 are copper oxide able to release monovalent copper ions and divalent copper ions, silver particles which be able to release silver ions in aqueous solution. The at least two precious metals 201 can also be the following metals but not for the limitation such as gold, palladium, platinum, aluminum, nickel, cobalt, zinc, the compounds of zinc, silicon, calcium, titanium, chromium, and the combination thereof The better choices are zinc and the compounds of zinc. Zinc and the compounds of zinc have a very good ability to kill the microbes/germs and fungus because they can destroy the structure of bacteria or produce hydrogen peroxide to achieve the purpose of killing bacteria. The absorber 300 can use the same materials as the at least one absorbent carbonaceous base 200. To reduce the cost, the absorber 300 can also use the active carbon fiber cloth, active carbon fiber paper, active carbon fiber felt and the combination thereof, or the absorber 300 can mix with or fill in the highly absorbent natural fiber such as cotton, linen, wool, man-made cellulose fiber, superabsorbent polymer, and the combination thereof The absorber 300 can carry a precious metal 201. The precious metal 201 comprises silver, gold, palladium, platinum, copper, the compounds of copper, aluminum, nickel, cobalt, zinc, the compounds of zinc, silicon, calcium, titanium, chromium, and the combination thereof The diameters of the particles of the precious metal 201 are less than 2000 nm. The best is less than 500 nm. The surface layer 100 of the tampon structure of the present invention can choose a water-permeable air-laid nonwoven, porous plastic membrane PE or PET, a spunlace nonwoven fabric, and the combination thereof. The rope 400 of the tampon structure of this invention is connected with the absorber 300 by following ways but not for limitation: the rope 400 is settled in the internal part of absorber 300, the rope 400 is sawed in the internal part of absorber 300, and after the rope 400 gets through one end of the absorber 300 ties a knot to avoid the rope 400 to falling off.

Some therapeutically active components are carried depending on needs in the surface layer 100, the absorber 300, or the at least one absorbent carbonaceous base 200 of the tampon structure of this present invention. The therapeutically active components can be following components but not for limitation: metronidazole, clindamycin, butoconazole, clotrimazle, miconazole, nystatin, tioconazole, terconazole, econazole, erythromycin, clindamycin, tetrachcline, cephalosporin, acrisorcin, haloprogin, iodochlorhydroxyquin, tolnaftate, triacetin, centella asiatica, econazole nitrate, mafenide, mupirocin, povidone iodine, and the combination thereof.

In this present invention, there are several ways to make the at least two precious metals 201 carried by and/or inserted in the at least one absorbent carbonaceous base 200, giving a following example to describe one of the ways. The active carbon fiber cloth is soaked in the concentration of 1 mole solution of the copper nitrate and silver nitrate for 1-720 minutes. The copper and silver particles are able to be carried by and/or inserted in the active carbon fiber. In this process, the pH value is between 3 and 8. The active carbon fiber cloth which the copper and silver particles are carried by and/or inserted in keeps being dry. The active carbon fiber cloth which the copper and silver particles are carried by and/or inserted in is replaced into a high-temperature oven, the temperature being 120-800° C. for 5-120 minutes. This process can make the diameters of the copper and silver particles less than 500 nm and spreading evenly on the active carbon fiber cloth. Afterward, the active carbon fiber cloth which the copper and silver particles are carried by and/or inserted in is washed with water to remove the excessive copper and silver particles. After the active fiber cloth keeps being dry, the process of the nanoscale copper and silver particles carried by and/or inserted in the active carbon fiber cloth is completed. The methods of making the at least two precious metals 201 carried by and/or inserted in the surface layer 100 or on the at least one absorbent carbonaceous base 200 can also be metal coating or electrochemistry.

In the present invention, the at least two precious metals 201 are carried by and/or inserted in the at least one absorbent carbonaceous base 200. The better choices of at least two precious metals 201 are copper and silver particles with the diameters less than 500 nm. The purpose of carrying the at least two precious metals 201 on the at least one absorbent carbonaceous base 200 is further raising the antibacterial and antifungal ability. In the following embodiments, the copper and silver particles are carried by or inserted in the at least one absorbent carbonaceous base 200 as the better embodiments. With the data from the laboratory, it accounts for the ability of inhibiting the growth of bacteria and fungus. The data from the laboratory is the report of testing the related materials provided by the inventors. The method of testing, the adopted microbes and the data of results are displaying as following.

In the embodiments below, the content of metals, the observation of carbon materials are using the following methods and instruments.

### The Test of the Content of Metals

The instruments of the test are the following:
   a. microwave Lab Station
   b. Inductively Coupled Plasma Optima Optical Emission Spectrometer, ICP-OES
The methods of the test are the following:
   1. The dried carbon material which the metals are carried by or inserted in weighed from 50 mg to 80 mg is put into the vessels of the microwave lab station, adding the solution comprising 1 ml Hydrochloric acid, 5 ml Sulfuric acid 1 ml Hydrofluoric acid, and 1 ml Nitric acid. Afterward, the vessels are sealed.
   2. The vessels should be arranged symmetrically (at least four vessels) into the microwave lab station, to carry on the procedure.
   3. After processing in the microwave lab station, the vessels are put for 3 to 4 hours. After the temperature of the vessels dropped to room temperature, they are taken out.
   4. The solution is filtered by the filter paper of number 40. The filtered solution is put into the polypropylene centrifuge bottle. The deionized water is added into the polypropylene centrifuge bottle to quantify the volume up to 50 ml. The solution and bottle keeps refrigerated.
   5. The standard solution of the metals specified for ICP-OES is prepared. The concentration of 0 ppm, 0.05 ppm, 0.1 ppm, 0.5 ppm, 1 ppm, 3 ppm and 5 ppm of standard solution are prepared for the calibration curves.
   6. All the test sample solution is tested by the ICP-OES. Every sample is tested for 3 to 5 times. The results are the average of the data. After each test, the sampling area of the machine must be washed several times by diluted nitric acid solution and deionized water to avoid the contaminating the following tests.

### The Observation of the Surface of the Carbon Materials

The instrument of the observation is following:
   Cold Field Emission Scanning Electron Microscope and Energy Dispersive Spectrometer
The methods of the observation are the following:
   The proper sized carbon material carrying the metals is fixed on the platform, with a diameter of 2.5 cm, by the adhesive carbon tape. Before the sample is observed by the microscope, the sample should be heated by the heat plate at 80° C., for one hour. The criteria include 10 kv to 15 kv accelerating voltage and 5000 to 400000 magnification for the surface observation of the carbon material carrying the metals. Because of the observation of the surface, the carbon material did not be coated with gold.

### The Test of Real Density

The instrument of the test is following:
   Accupyc 1330 Pycnometer
The methods of the test are the following:
   The dried sample is put into the container of the Accupyc 1330 Pycnometer and weighed the sample. The pressured helium gas is released into the test chamber of the machine After it reaches the balanced situation, the volume of the sample can be calculated by the ideal gas equation, PV=nRT, and the average of the real density of the sample can be obtained.

### The Surface Area Analysis (Brunauer-Emmett-Teller Method, BET)

The instrument of the analysis is the following:
   Micromeritics ASAP2020
The methods of the analysis are the following
   After the sample is heated and degas, the nitrogen gas is filled in. The temperature and pressure are fixed at 77 k and 760 mm-Hg. A BET value is then obtained.

### Embodiment 1

The material is the active carbon fiber cloth as the at least one absorbent carbonaceous base 200 and the BET value is 1050 m²/g. The active carbon fiber cloth is soaked in the 0.01M solution of the silver nitrate and cooper nitrate and mixed under 50 rpm rotation for 1 hour. The cloth is dehydrated and replaced in a 120° C. oven to get rid of the liquid.

The soaked active carbon fiber cloth is protected under nitrogen gas. After the temperature is raised from room temperature to 600° C. at a rate of 4° C./min, the thermocracking is being carried out for 1 minute. Afterward, the temperature is declined at a rate of 10° C./min until the temperature dropping to room temperature.

The active carbon fiber cloth is washed for 2 hours and dried in the oven at 120° C. for 2 hours. The active carbon fiber cloth which the copper and silver particles are carried by and/or inserted in is obtained. As the aforementioned methods for testing, the results show that the content of silver in the active carbon fiber cloth is 0.06 wt % of total weight. The silver particles are in the range of nanoscale, with the diameters of 10 to 50 nm. The content of copper is 0.05 wt % of total weight. The copper particles are in the range of nanoscale, with the diameters of 100 to 500 nm.

The real density of copper and silver particles carried by and/or inserted in the active carbon fiber cloth is 2.08 g/cm³. The content of carbon is 85.5 wt %, the content of oxygen is 10.4 wt % and the BET value is 1032 m²/g.

The tampon structure is made by the described active fiber cloth which the copper and silver particles are carried by and/or inserted in. The surface layer 100 of it is the porous plastic PE membrane, and it is s affixed with the active carbon fiber cloth which the copper and silver particles are carried by and inserted in by the acrylic adhesive for medical use. The absorber 300 is adopted phenolic active carbon fiber felt.

The ability of antibacteria and antifungus of the tampon structure made by described methods and materials is tested.

### Embodiment 2

The phenolic active carbon fiber felt with the BET value 1420 m²/g as the material of the at least one absorbent carbonaceous base 200 is adopted. The same methods as mentioned in embodiment 1 are adopted. The results showed that the content of silver is 0.07 wt % of the total weight, and the diameters of silver particles are about 50 to 200 nm. The content of copper is 0.04 wt % of the total weight. The copper particles are in the range of nanoscale and the diameters of copper particles are about 100 to 500 nm.

The real density of the copper and silver particles carried by and/or inserted in the active carbon fiber felt is 2.04 g/cm³, the content of carbon is 85.5 wt %, the content of oxygen is 13.2 wt %, the BET value is 1380 m²/g.

The tampon structure is made by the described phenolic active carbon fiber felt which the copper and silver particles are carried by and/or inserted in. The surface layer 100 of it is the porous plastic PE membrane, and it is affixed with the phenolic active carbon fiber felt which the copper and silver particles are carried by and inserted in by the acrylic adhesive for medical use. The absorber 300 is also adopted the phenolic active carbon fiber felt. The ability of antibacteria and antifungus of the tampon structure made by described methods and materials is tested.

### Embodiment 3

Fireproof-fiber felt is adopted. It is activated under 800° C. vapor for 10 minutes to make polyacrylonitrile active carbon felt with BET value 632 m²/g. As the polyacrylonitrile active carbon felt is adopted as the material of the at least one absorbent carbonaceous base 200, it is soaked in the 0.01 M solution of silver nitrate and copper nitrate under vacuum for 5 hours. Afterward, the polyacrylonitrile active carbon felt is dehydrated and liquid is removed by the 120° C. oven for 2 hours. The soaked polyacrylonitrile active carbon felt is settled in to the 400° C. high-temperature oven, for the 90-minute thermocracking. The silver and copper particles carried by and/or inserted in the polyacrylonitrile active carbon felt is washed at the rate 4.5 litters/minute of water flow for 3 hours and dried in the 120° C. oven for 2 hours. The final product is tested and the results showed that the content of silver is 0.04 wt % of total weight, and the diameters of the silver particles are 50 to 200 nm The content of copper is 0.02 wt % of total weight, the copper particles are in the range of nanoscale and the diameters are 100 to 500 nm.

The real density of the copper and silver particles carried by and/or inserted in the polyacrylonitrile active carbon felt is 1.93 g/cm³. The content of carbon is 86.0 wt %. The content of oxygen is 10.3 wt %, and the BET value is 589 m²/g.

The tampon structure is made by the described polyacrylonitrile active carbon felt which the copper and silver particles are carried by and/or inserted in. The surface layer 100 of it is the porous plastic PE membrane, and it is affixed with the polyacrylonitrile active carbon felt which the copper and silver particles are carried by and inserted in by the acrylic adhesive for medical use. The absorber 300 is also adopted the phenolic active carbon fiber felt. The ability of antibacteria and antifungus of the tampon structure made by described methods and materials is tested.

### Control Group 1

As the active carbon fiber cloth is the material of the at least one absorbent carbonaceous base 200 with the BET value 1050 m²/g, it is soaked in the 0.01M silver nitrate solution and mixed with 50 rpm for 2 hours, afterward, the soaked active carbon fiber cloth is dehydrated and liquid is removed by 120° C. oven for 2 hours.

As the soaked active carbon fiber cloth is under the protection of nitrogen gas, at 4° C./minute-rate of raising temperature, the temperature is raised from room temperature to 600° C. for 1 hour of thermocracking. Afterward, the temperature is declined at a rate of 10° C./minute until to the room temperature.

The silver particles carried by or inserted in the active carbon fiber is washed for 2 hours and dried for 120° C. for 2 hours. The final product is obtained and tested with described methods. The results showed that the content of silver is 0.10 wt % of the total weight and the silver particles on the active carbon fiber are in the range of nanoscale. The diameters of silver particles are 10 to 50 nm.

The real density of the silver particles carried by and/or inserted in the active carbon fiber cloth is 2.07 g/cm³. The content of carbon is 85.6 wt %. The content of oxygen is 10.5 wt %, and the BET value is 1036 m²/g.

The tampon structure is made by the described active carbon fiber cloth which the silver particles are carried by and/or inserted in. The surface layer 100 of it is the porous plastic PE membrane, and it is affixed with the active carbon fiber cloth which the silver particles are carried by and inserted in by the acrylic adhesive for medical use. The absorber 300 is also adopted the phenolic active carbon fiber felt. The ability of antibacteria and antifungus of the tampon structure made by described methods and materials is tested.

### Control Group 2

As the active carbon fiber cloth is the material of the at least one absorbent carbonaceous base 200 with the BET value 1050 m²/g, it is soaked in the 0.01M copper nitrate solution and mixed with 50 rpm for 5 hours. Afterward, the soaked active carbon fiber cloth is dehydrated and liquid w is removed by 120° C. oven for 2 hours.

As the soaked active carbon fiber cloth is under the protection of nitrogen gas, at 4° C./minute -rate of raising temperature, the temperature is raised form room temperature to 600° C. for 1 hour of thermocracking. Afterward, the temperature is declined at a rate of 10° C./minute until to the room temperature.

The copper particles carried by and/or inserted in the active carbon fiber is washed for 2 hours and dried for 120° C. for 2 hours. The final product is obtained and tested with described methods. The results showed that the content of copper is 0.18 wt % of total weight and the copper particles on the active carbon fiber cloth are in the range of nanoscale. The diameters of copper particles are 100 to 500 nm.

The real density of the copper carried by and/or inserted in the active carbon fiber cloth is 2.08 g/cm³. The content of carbon is 85.3 wt %. The content of oxygen is 10.6 wt %, and the BET value is 1032 m²/g.

The tampon structure is made by the described active carbon fiber cloth which the copper particles are carried by and/or inserted in. The surface layer 100 of it is the porous plastic PE membrane, and it is affixed with the active carbon fiber cloth which the copper particles are carried by and inserted in by the acrylic adhesive for medical use. The absorber 300 is also adopted the phenolic active carbon fiber felt. The ability of antibacteria and antifungus of the tampon structure made by described methods and materials is tested.

### Control Group 3

The active carbon fiber cloth is adopted as the material of the at least one absorbent carbonaceous base 200 with BET value 1050 m²/g. The real density is 2.07 g/cm³, the content of carbon is 85.0 wt %, and the content of oxygen is 10.3 wt %.

The tampon structure is made by the described active fiber cloth. The surface layer 100 of it is the porous plastic PE membrane, and the surface layer 100 w is affixed with the active carbon fiber cloth by the acrylic adhesive for medical use. The absorber 300 is adopted the phenolic active carbon fiber felt.

The described tampon structure with aforementioned combination is made by the same methods as mentioned in embodiment 1. The ability of antibacteria and antifungus of the tampon structure made by described methods and materials is tested.

TABLE 1
Results of the tests for Candida albicans

| | | RESULTS | | | | | |
|---|---|---|---|---|---|---|---|
| | | Control | Control | Control | | | |
| TESTS | | group 1 | group 2 | group 3 | Embodiment 1 | Embodiment 2 | Embodiment 3 |
| Candida | B (CFU/mL) | 1.95E+5 | 1.96E+5 | 1.96E+5 | 1.96E+5 | 1.96E+5 | 1.96E+5 |
| albicans | A (CFU/mL) | 2.28E+4 | 8.86E+3 | 2.28E+5 | <1 | <1 | <20 |
| | R (%) | 88.36 | 95.45 | <0 | >99.99 | >99.99 | >99.98 |

The bacteria concentration of shaken bacterial media (A): the bacteria concentration of bacterial media after shaking for one-hour

The initial concentration of bacterial media (B): the bacteria concentration of bacterial media after shaking for one-minute

The rate of reduction (R): R=100(B-A)/B

2.0E+2 representing 200, 1.3E+4 representing 13000; and so on

The weight of every sample is 0.1 g

TABLE 2
Results of the tests for Staphyloccocus aureus and Klebsiella pneumoniae

| | | RESULTS | | |
|---|---|---|---|---|
| | | Control | Control | |
| | | group | group | Embodiment |
| TESTS | | 1 | 2 | 1 |
| Escherichia | The initial number of | 1.13E+5 | 1.13E+5 | 1.13E+5 |
| coli | bacteria (B) | | | |
| | The post culturing | 2.55E+3 | 3.59E+4 | <20 |
| | number of bacteria (A) | | | |
| | The rate of reduction (R) | 97.74 | 68.11 | >99.98 |
| | (%) | | | |
| Klebsiella | The initial number of | 1.16E+5 | 1.16E+5 | 1.16E+5 |
| pneumoniae | bacteria (B) | | | |
| | The post culturing | 1.68E+3 | 2.54E+4 | <20 |
| | number of bacteria (A) | | | |
| | The rate of reduction | 98.55 | 78.10 | >99.98 |
| | (R)(%) | | | |

The initial number of bacteria (B): the number of bacteria of which the test sample and the stander sample once contacted with the bacteria media and then washed with zero the culturing time. If there is no significant difference in between both the described samples, the result is the average of them; if there is difference in between them, the larger number is the result.

The post culturing number of bacteria(A): the test sample is cultured at 37° C.,

18 to 24 hours.

The rate of reduction (R): R=100(B-A)/B

2.0E+2 representing 200, 1.3E+4 represents13000; and so on

The rate of reduction between 0<R≦100, representing an effective ability of killing microbe/germs.

The rate of reduction is less than 0, representing a null ability of killing microbe/germs.

TABLE 3
Results of the tests for Staphyloccocus aureus
Results for inhibiting Staphyloccocus aureus

| | | | | The average |
|---|---|---|---|---|
| | 1 | 2 | 3 | of three |
| Embodiment3 | 95.98% | 93.87% | 94.83% | 94.89% |
| Embodiment2 | 99.99% | 99.99% | 99.99% | 99.99% |

TABLE 4
Results of the tests for Escherichia coli

| | | RESULTS | | | | | |
|---|---|---|---|---|---|---|---|
| | | Control | Control | Control | | | |
| TESTS | | group 1 | group 2 | group 3 | Embodiment 1 | Embodiment 2 | Embodiment 3 |
| Escherichia | B | 2.66E+5 | 2.55E+5 | 2.55E+5 | 2.52E+5 | 2.64E+5 | 2.64E+5 |
| coli | (CFU/mL) | | | | | | |
| | A | 4.43E+2 | 1.36E+4 | 8.87E+2 | <1 | <1 | <1 |
| | (CFU/mL) | | | | | | |
| | R (%) | 99.8 | 94.67 | 99.65 | >99.99 | >99.99 | >99.99 |

The bacteria concentration of shaken bacterial media (A): the bacteria concentration of bacterial media after shaking for one-hour

The initial concentration of bacterial media (B): the bacteria concentration of bacterial media after shaking for one-minute

The rate of reduction (R): R=100(B-A)/B

2.0E+2 representing 200, 1.3E+4 representing 13000; and so on

The weight of every sample is 0.2 g.

One of the most infected reasons for vaginitis is the infection of Candida albicans. The most common symptoms are extremely itchy of vagina and external pudendal area, pain during urination, white and sticky vaginal discharge. The results of tests for Candida albicans showed in the Table 1. In the control group 1, whereas the reduction rate of bacteria of the active carbon fiber cloth which the silver particles are carried by and/or inserted the active carbon fiber cloth is 88.36%, in the control group 2, the reduction rate of bacteria of the active carbon fiber cloth which the copper particles are carried by and/or inserted in the is 95.45%. In the embodiment 1, 2 and 3, the reduction rate of bacteria of the active carbon fiber cloth which the copper and silver particles are carried by and/or inserted in is above 99.9%. This accounts for the absolute effect on killing Candida albicans. This result demonstrates that the active carbon fiber cloth which the copper and silver particles are carried by and/or inserted in simultaneously keeps a better effect of killing Candida albicans than the active carbon fiber cloth which only the copper particles are carried by and/or inserted in. The active carbon fiber cloth which only the copper particles are carried by and/or inserted in keeps a better effect of killing Candida albicans than the active carbon fiber cloth which only the silver particles are carried by and/or inserted in.

Staphyloccocus aureus causes various purulent infections, urinary tract infections and toxic shock syndrome from the infection of vagina during females' menstrual period. The table 2 and Table 3 demonstrate the results of Staphyloccocus aureus tests. In the embodiment 1 and 2, the active carbon fiber cloth which the copper and silver particles are simultaneously carried by and/or inserted in demonstrates the absolute effect of killing bacteria.

Klebsiella pneumonia often causes the patients with defective immune system liver abscess, urinary tract infection, septicemia and cerebromeningitis. The results of Klebsiella pneumonia in the Table 2 demonstrate the active carbon fiber carrying both silver and copper particles has the ability to kill bacteria completely.

Being the normal flora in human intestine, E. coli can cause various infections after invading human body such as acute gastroenteritis, urinary tract infection, cerebromeningitis and pneumoniae. The results in the Table 4 are the tests of E. coli. The active carbon fiber cloth carrying silver and copper particles has the ability to kill bacteria completely.

From described results of tests, the reduction of killing microbes of the active carbon fiber cloth carrying both silver and copper particles, whether in the performance of killing fungus or bacteria, is better than the active carbon fiber cloth only carrying silver particles or copper particles individually.

To describe the embodiments in conjunction with the following figures, the present invention can be illustrated with more details. However, the following figures are for demonstration, not be drawn in accordance with the actual proportion.

In the FIG. 1, the cross-section diagram belongs to one embodiment of the tampon structure in the present invention. The tampon structure comprises the surface layer 100, the at least one absorbent carbonaceous base 200 carrying the at least two precious metals 201, the absorber 300 containing a superabsorbent polymer 301 and the rope 400 connecting to the absorber 300. The surface layer 100 is porous, allowing fluid flow through the surface layer 100 into the at least one absorbent carbonaceous base 200. The better material for the at least one absorbent carbonaceous base 200 includes active carbon, active carbon fiber, porous graphite, flexible graphite, expandable graphite, and the combination thereof The better type for the at least one absorbent carbonaceous base 200 is active carbon fiber. A way to use the tampon structure is putting it into vagina and the rope 400 dangles from the opening of vagina to outside the body. Holding the rope 400 and pulling it toward outside the body, the tampon structure can be removed and the absorber 300 is also removed out and then discarded. The absorber 300 can use the active carbon material, carbon material blended with natural fiber, synthetic fiber, antibacterial nature fiber or synthetic fiber.

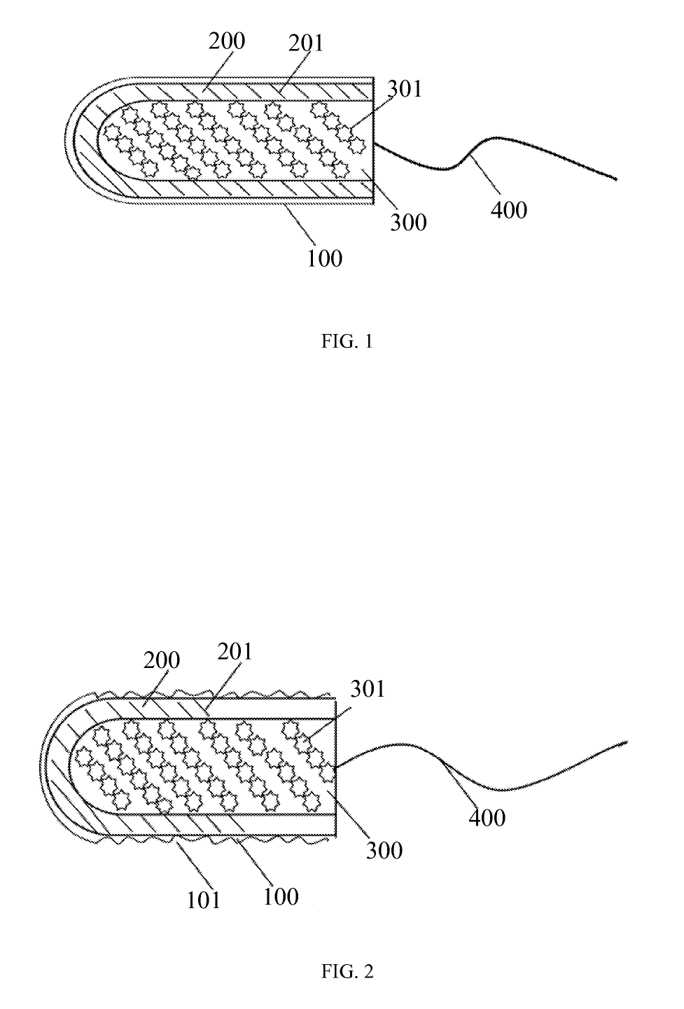

In the FIG. 2, the cross-section diagram belongs to another embodiment. The tampon structure comprises the surface layer 100, the at least one absorbent carbonaceous base 200 carrying the at least two precious metals 201, the absorber 300 containing the superabsorbent polymer 301, and the rope 400 connecting to the absorber 300. In the tampon structure, a groove 101 is settled on the surface structure 100, to assist the fluid flow through the surface layer 100. The at least one absorbent carbonaceous base 200 covers one part of the absorber 300 but not cover the absorber 300 completely.

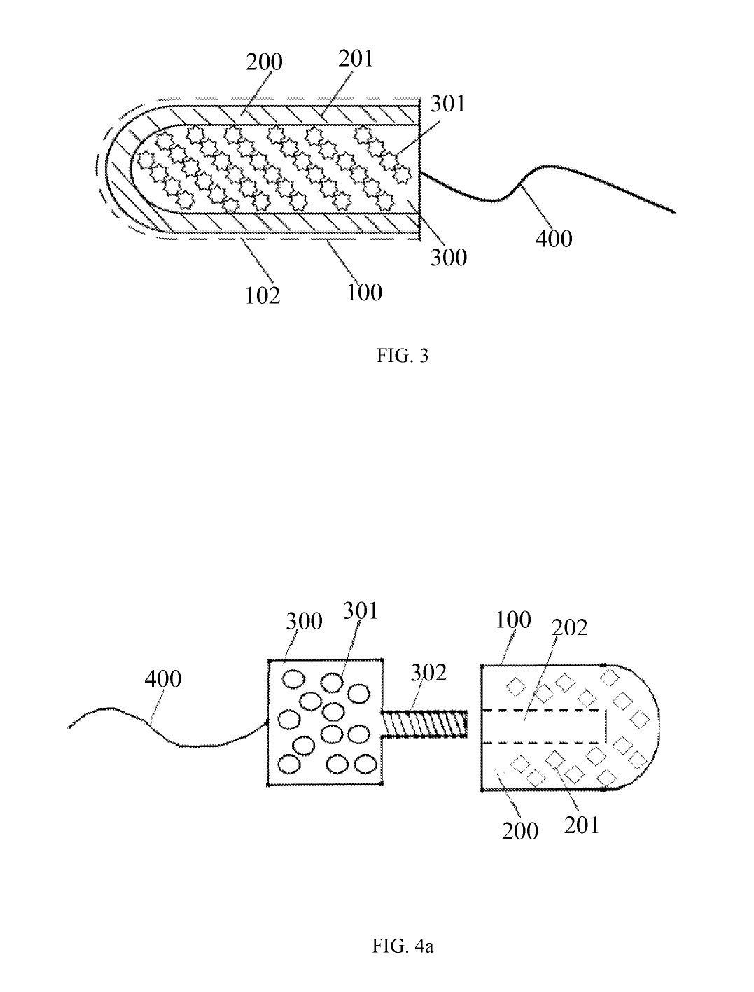

In the FIG. 3, the cross-section diagram belongs to another embodiment. The tampon structure comprises the surface layer 100, the at least one absorbent carbonaceous base 200 carrying the at least two precious metals 201, the absorber 300 containing the superabsorbent polymer 301, and the rope 400 connecting to the absorber 300. In the tampon structure, there are pores 102 on the surface layer 100 to increase the permeability of the surface layer 100 to the fluid.

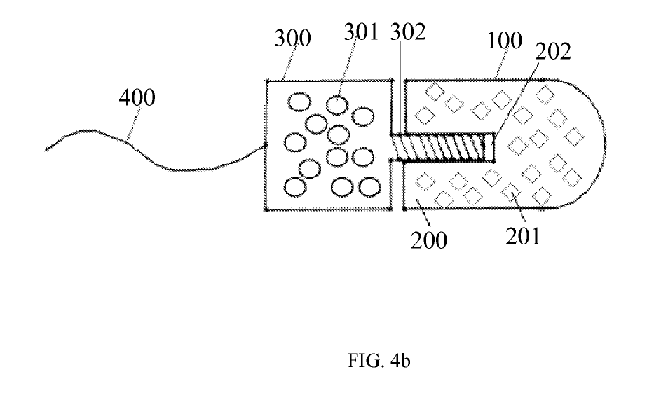

In the FIGS. 4a and 4b, the cross-section diagram belongs to the other embodiment. Referring to the FIG. 4a, the tampon structure comprises the surface layer 100, a hollow screw-thread track 202 being settled on the at least one absorbent carbonaceous base 200 which also carrying the at least two precious metals 201, a prominent bolt 302 being settled on the absorber 300 which also containing the superabsorbent polymer 301, and the rope 400 connecting to the absorber 300. The at least one absorbent carbonaceous base 200 possessing the hollow screw-thread track 202 and carrying the at least two precious metals 201 can be used alone or through matching the bolt 302 with the hollow screw-thread track 202, the at least one absorbent carbonaceous base 200 being able to connected with the absorber 300 can be used together. It can be used in either way depending on the needs of a user.

Referring to the FIG. 4b, if it is not in the menstrual period, the user can use the at least one absorbent carbonaceous base 200 alone. If it is in the menstrual period, the at least one absorbent carbonaceous base 200 and the absorber 300 can be used together after connecting tightly to each other. The end of the bolt 302 is in pyramidal shape and there is a pyramidal cavity on the corresponding end of the hollow screw-thread track 202. That makes the bolt 302 and the hollow screw-thread track 202 can be connected tightly. Consequently, the absorber 300 and the at least one absorbent carbonaceous base 200 cannot be separated easily. It is easily be used. The better material for the at least one absorbent carbonaceous base 200 includes active carbon, active carbon fiber, porous graphite, flexible graphite, expandable graphite, and the combination of thereof. The better type for the at least one absorbent carbonaceous base 200 is active carbon fiber. The way to use the tampon structure is putting it into vagina and the rope 400 dangles from the opening of vagina to outside the body. Holding the rope 400 and pulling it toward outside the body, the tampon structure can be removed and the absorber 300 is also removed out and then discarded.

Not being the limitation to the present invention, the described disclosure has demonstrated the present invention. Anyone being acquaintance with the related field, within the range of the present invention and within the spirit of this invention, can modify and change it. The protection of this present invention is defined by the following claims.

What is claimed is:

1. A tampon structure, comprising:
an absorber;
at least one absorbent carbonaceous base, the absorbent carbonaceous covering at least one part of the absorber;
at least two precious metals comprise copper particles and silver particles, and being carried by and/or inserted in the at least one part of the at least one absorbent carbonaceous base; and
a surface layer, covering the at least one part of the at least one absorbent carbonaceous base.

2. The tampon structure as claimed in claim 1, wherein the surface layer comprises a water-permeable air-laid nonwoven, porous plastic membrane PE or PET, a spunlace nonwoven fabric and the combination thereof

3. The tampon structure as claimed in claim 1, wherein a groove is settled on the surface layer.

4. The tampon structure as claimed in claim 1, wherein the at least one absorbent carbonaceous base comprises active carbon, active carbon fiber, flexible graphite, expandable graphite, cloth, paper, felt, nonwoven and the combination thereof.

5. The tampon structure as claimed in claim 1, wherein the at least two precious metals comprise silver, gold, palladium, platinum, copper, aluminum, nickel, cobalt, zinc, the compounds of zinc, silicon, calcium, titanium, chromium, and the combination thereof.

6. The tampon structure as claimed in claim 1, wherein diameters of the particles of the at least two precious metals are less than 2000 nanometers.

7. The tampon structure as claimed in claim 1, wherein the absorber comprise highly absorbent natural fiber, comprising cotton, linen, wool, man-made cellulose fiber, active carbon fiber cloth, active carbon fiber paper, active carbon fiber felt, superabsorbent polymer, and the combination thereof.

8. The tampon structure as claimed in claim 1, wherein the surface layer, the absorber, or the at least one absorbent carbonaceous base further carries at least one therapeutically active component.

9. The tampon structure as claimed in claim 8, wherein the therapeutically active component comprises metronidazole, clindamycin, butoconazole, clotrimazle, miconazole, nystatin, tioconazole, terconazole, econazole, erythromycin, tetrachcline, cephalosporin, acrisorcin, haloprogin, iodochlorhydroxyquin, tolnaftate, triacetin, centella asiatica, econazole nitrate, mafenide, mupirocin, povidone iodine, and the combination thereof.

10. The tampon structure as claimed in claim 1, wherein the tampon structure further comprises a rope.

11. The tampon structure as claimed in claim 1, wherein the absorber carries a precious metal.

12. The tampon structure as claimed in claim 11, wherein the precious metal comprises silver, gold, palladium, platinum, copper, the compounds of copper, aluminum, nickel, cobalt, zinc, the compounds of zinc, silicon, calcium, titanium, chromium, and the combination thereof.

13. The tampon structure as claimed in claim 12, wherein diameters of particles of the precious metal are less than 2000 nanometers.

14. The tampon structure as claimed in claim 1, wherein the tampon structure is column shape or strip shape.

15. A tampon structure comprises:
at least one absorbent carbonaceous base with a hollow screw-thread track;
at least two precious metals carried by and/or inserted in the at least one part of the at least one absorbent carbonaceous base and comprising copper particles, silver particles, gold, palladium, platinum, aluminum, nickel, cobalt, zinc, silicon, calcium, titanium, chromium, the compound of zinc and the combination thereof; and
a surface layer covering at least one part of the absorbent carbonaceous base.

16. The tampon structure as claimed in claim 15, wherein the tampon structure further comprises:
an absorber; and
a prominent bolt being settled on the absorber, the bolt being match to the hollow screw-thread track in the at least one absorbent carbonaceous base; the absorber and the at least one absorbent carbonaceous base being connected through the bolt and the hollow screw-tread track for needs.

17. The tampon structure as claimed in claim 15, wherein the surface layer comprises a water-permeable air-laid nonwoven, porous plastic membrane PE/PET, a spunlace nonwoven fabric and the combination thereof.

18. The tampon structure as claimed in claim 15, wherein a groove is settled on the surface layer.

19. The tampon structure as claimed in claim 15, wherein the at least one absorbent carbonaceous base comprises active carbon, active carbon fiber, flexible graphite, expandable graphite, cloth, paper, felt, nonwoven, and the combination thereof.

20. The tampon structure as claimed in claim 15, diameters of the particles of the at least two precious metals are less than 2000 nanometers.

21. The tampon structure as claimed in claim 15, wherein the absorber comprises highly absorbent natural fiber, comprising cotton, linen, wool, man-made cellulose fiber, active carbon fiber cloth, active carbon paper, active carbon felt, superabsorbent polymer, and the combination thereof.

22. The tampon structure as claimed in claim 15, wherein the surface layer, the absorber, or the at least one absorbent carbonaceous base further carries at least one therapeutically active component.

23. The tampon structure as claimed in claim 22, wherein the therapeutically active component comprises metronidazole, clindamycin, butoconazole, clotrimazle, miconazole, nystatin, tioconazole, terconazole, econazole, erythromycin, tetrachcline, cephalosporin, acrisorcin, haloprogin, iodochlorhydroxyquin, tolnaftate, triacetin, centella asiatica, econazole nitrate, mafenide, mupirocin, povidone iodine, and the combination thereof.

24. The tampon structure as claimed in claim 15, wherein the tampon structure further comprises a rope.

25. The tampon structure as claimed in claim 15, wherein the absorber carries a precious metal.

26. The tampon structure as claimed in claim 25, wherein the precious metal comprises silver, gold, palladium, platinum, copper, the compounds of copper, aluminum, nickel, cobalt, zinc, the compounds of zinc, silicon, calcium, titanium, chromium, and the combination thereof.

27. The tampon structure as claimed in claim 26, wherein the precious metal comprises copper particles, the compounds of copper particles and the combination thereof with diameters of the particles less than 500 nanometers.

28. The tampon structure as claimed in claim 15, wherein the tampon is column shape or strip shape.

29. The tampon structure as claimed in claim 15, wherein the at least one absorbent carbonaceous base is blended with natural fiber and synthetic fiber.

30. The tampon structure as claimed in claim 15, wherein the surface area analysis by using Brunauer-Emmett-Teller (BET), the BET value oft being above 400 m²/g.

31. The tampon structure as claimed in claim 15, wherein true density of the at least one absorbent carbonaceous base is above 1.7 g /cm³.
